(19) United States
(12) Patent Application Publication
(10) Pub. No.: US 2011/0230398 A1
(43) Pub. Date: Sep. 22, 2011

(54) Apolipoprotein C-II or its active fractions for the prevention or treatment of obesity and related metabolic disorders

(76) Inventor: Surendra K. Gupta, Laguna Niguel, CA (US)

(21) Appl. No.: 12/727,339

(22) Filed: Mar. 19, 2010

(51) Int. Cl. A61K 38/00; C07K 14/00; C07K 2/00; A61P 3/04

(52) U.S. Cl. 514/4.8; 530/350; 530/300; 514/6.9; 514/7.4

(57) ABSTRACT

Apoliporotein C-II or any active fraction of its amino acid sequence that activates lipoprotein lipase (LPL) in mammals. Increase in activity of LPL can increase expenditure of energy, and therefore an activator of LPL can be an effective agent for the prevention or treatment of obesity or related metabolic disorders, including cardiovascular disease, diabetes and hypertension in mammals including domestic pets and humans. Apolipoprotein C-II or its active fractions can be used as a diet supplement in the form of fortification to a natural product such as milk, cereal, beverage; as a nutritional supplement; or as a therapeutic agent in conventional forms of administration.

### FIELD OF THE INVENTION

The present invention relates to apolipoprotein C-II and an active fraction of its composition which can be used for prevention or treatment of obesity or related metabolic disorders, including cardiovascular disease, diabetes and hypertension in mammals including domestic pets and humans. Apolipoprotein C-II or its active fractions can be used in the form of fortification to a natural product such as milk, cereal, beverage; as a nutritional supplement; or as a therapeutic agent in conventional forms of administration.

### BACKGROUND OF THE INVENTION

Obesity is now recognized by the medical profession as a metabolic disease. In the USA and some European countries, it is estimated that 25% of the adult population is considered clinically obese (Body Mass Index>30). Obesity can be a debilitating condition that reduces the quality of life and increases the risk of related disorders such as diabetes, cardiovascular disease and hypertension. It has been estimated that $45 billion of U.S. healthcare costs, or 8% per annum of total healthcare spending, is a direct result of obesity. The traditional approaches to long-term weight management, such as diet and exercise, have proved ineffective alone in controlling the spread of obesity. Today, more than ever, there is considerable interest in developing safe, effective drugs for the treatment of obesity.

There are several approaches which are currently in progress for the development of such drugs for obesity.

One approach includes drug candidates that block the cannabinoid receptors, boost hypothalamus hormones, activate serotonin receptors, or function as monoamine reuptake inhibitors. This approach affects appetite metabolism but could have serious side effects including psychiatric disorders.

A second approach is the reduction of energy intake and thus reduction of the body's ability to digest and absorb food, in particular fat. The key enzymes involved in the digestion of fat are lipases. The currently-marketed drug Orlistat works by inhibiting pancreatic lipase, which in turn inhibits the breakdown of triglycerides so that fat is not absorbed by the body and is excreted undigested as waste product. Recently, based upon U.S. Pat. No. 6,624,161, an improved lipase inhibitor called Cetilistat was developed to treat obesity, and is now in Phase III clinical trial. However, similar to Orlistat, it produces side effects such as oily, loose stools, fecal incontinence, frequent bowel movements, and flatulence.

A third possible approach is to increase the body's energy expenditure by developing an activator that increases lipoprotein lipase (LPL) activity or synthesizes more molecules of LPL, which will increase fat metabolism and thereby decrease weight or prevent weight gain.

Recently, it has been noted that compound NO-1886 (ibrolipim) increases the synthesis of LPL, which causes a decrease in triglyceride levels, an increase in high-density lipoprotein (HDL) cholesterol, and the prevention of fat accumulation in high-fat-fed rats. This suggests that activation of LPL may play a significant role in prevention and treatment of obesity and related metabolic disorders.

In an unrelated study involving the long-term effects of several diets on weight loss (including a high fat/low carbohydrate diet, a high carbohydrate/low fat diet, and a mediterranean diet), it was shown that a high fat/low carbohydrate diet produced higher weight loss than the other two diets. Although the results could not be satisfactorily explained, a possible explanation may be that a high fat diet induces activation of LPL and related enzymes during lipolysis, and therefore causes energy expenditure and weight loss. Furthermore, applicant has observed that patients on a high fat/low carbohydrate diet produce high amounts (1-8 mmoles) of β-hydroxybutyrate in their blood, which is likely to be the case when either LPL is activated or more molecules of LPL are synthesized, and triglyceride from fat is broken down by LPL (unpublished data).

Based upon the above data, it seems reasonable to conclude that substances that activate either LPL activity or the gene that causes additional synthesis of LPL, could potentially be used for prevention or treatment of obesity and related metabolic disorders.

### SUMMARY OF THE INVENTION

It is an object of the present invention to find activators of lipoprotein lipase (LPL), which can be used for prevention or treatment of obesity and related metabolic disorders. Throughout this text, the prevention and/or treatment of any disorder means any effect that mitigates any damage or any medical disorder to any extent, and includes prevention and treatment themselves. The term “treatment” means any amelioration of disorder, disease, syndrome, condition, pain, or a combination of two or more thereof.

It is another object of the present invention to find a naturally occurring substance that can function as an activator of LPL and can be used for prevention or treatment of obesity and related metabolic disorders.

It is a further object of the present invention to employ apolipoprotein C-II (Apo C-II) as a naturally occurring substance that can be used for prevention or treatment of obesity and related metabolic disorders.

It is an additional object of the invention to use an active fraction amino acid sequence of Apo C-II that is as equally active as Apo C-II, and can be used for prevention or treatment of obesity and related metabolic disorders.

It is a further object of the invention to have a food, beverage or drug preparation containing Apo C-II or its active fraction that can be used for prevention or treatment of obesity and related metabolic disorders.

### DETAILED DESCRIPTION OF THE INVENTION

In the search to find compounds or substances found in nature which can function as an activator of lipoprotein lipase (LPL), the applicant discovered a naturally occurring substance found in milk and blood that activates LPL, namely apolipoprotein C-II (Apo C-II). The potential application and value of Apo C-II as an activator of LPL for prevention or treatment of obesity and related metabolic disorders, has not been known to those who are familiar in the art. Throughout this text, the prevention and/or treatment of any disorder means any effect that mitigates any damage or any medical disorder, to any extent, and includes prevention and treatment themselves. The term “treatment” means any amelioration of disorder, disease, syndrome, condition, pain, or a combination of two or more thereof.

Apo C-II is a known protein, comprised of 79 amino acids, with a molecular weight of about 8,800. The amino acid composition of Apo C-II is well defined and known. Furthermore, it is also known that some active fraction amino acid sequences, such as 55-79 amino acids from —COON terminal of Apo C-II, have the maximum Apo C-II activity. Therefore, any active fraction composition of Apo C-II, such as a peptide sequence of 55-79 amino acids, can be used instead of Apo C-II itself.

As Apo C-II is rich in skimmed milk and human and animal blood, it can be isolated from milk, serum or any other source rich in Apo C-II, and purified by well known purification methodologies. Apo C-II purified from human serum is also commercially available. As Apo C-II has a molecular weight of 8,800, it elutes from an ultrafiltration method using a 10,000 molecular weight (MW) membrane and contains Apo C-II. This is further concentrated using an ultrafiltration membrane of 1,000 and 3,500 MW cut-off. The partially purified Apo C-II can be stored in liquid form or can be lyophilized.

Apo C-II or its active fraction, such as the 55-79 amino acid sequence, has also been synthesized by known methodologies for peptide synthesis.

Apo C-II or its active fraction, according to the present invention, are primarily for use in relation to the prevention and/or treatment of obesity, but can also be used for treatment of hyperlipidaemia and related diseases such as hyperglycemia (type II diabetes), hypertension, cardiovascular disease, stroke, gastrointestinal disease and gastrointestinal conditions. The present invention also relates to non-medical weight loss, such as cosmetic weight loss, and includes improving bodily appearance in general.

Clearly, an important application of the invention is in relation to weight loss (of all kinds, as described above) in humans. However, the invention applies to medical and non-medical weight loss in any animal. Thus, the invention has veterinary application and is particularly useful in relation to medical and non-medical weight loss in companion animals such as pet cats and dogs, as well as in animals which provide meat for human consumption.

Apo C-II or its active fraction may be administered by any convenient method: oral (including by inhalation), parenteral, mucosal (e.g., buccal, sublingual, nasal), rectal, vaginal, or transdermal administration, and the compositions adapted accordingly.

For oral administration, the compounds can be formulated as liquids or solids, e.g., drops, gels, powders, solutions, syrups, suspensions or emulsions, tablets, capsules and lozenges. It can also be supplied as fortified milk, where Apo C-II or its active fraction is added back to skimmed milk or whole milk and further homogenized if necessary.

Compositions for oral (or nasal) administration may also be formulated as aerosols. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve intended for disposal once the contents of the container have been exhausted. Where the dosage form comprises an aerosol dispenser, it will contain a pharmaceutically acceptable propellant. The aerosol dosage forms can also take the form of a pump-atomizer.

A composition in the form of a capsule for oral administration can be prepared using routine encapsulation procedures. For example, powders, granules or pellets containing the active ingredient can be prepared using standard carriers and then filled into a hard gelatin capsule; alternatively, a dispersion or suspension can be prepared using any suitable pharmaceutical carrier(s), e.g., aqueous gums, celluloses, silicates or oils, and the dispersion or suspension then filled into a soft gelatin capsule.

Compositions for oral administration may be designed to protect the active ingredient against degradation as it passes through the alimentary tract, perhaps by an outer coating of the formulation on a tablet or capsule.

Typical parenteral compositions consist of a solution or suspension of the compound in a sterile aqueous or non-aqueous carrier or parenterally acceptable oil, e.g., polyethylene glycol, polyvinyl pyrrolidone, lecithin, arachis oil or sesame oil. Alternatively, the solution can be lyophilized and then reconstituted with a suitable solvent just prior to administration.

Compositions suitable for mucosal administration (buccal, sublingual or nasal) include aerosols (see above), tablets, lozenges and pastilles, wherein the active ingredient is formulated with a carrier such as sugar and acacia, or gelatin and glycerin.

Compositions for rectal or vaginal administration are conveniently in the form of suppositories (containing a conventional suppository base such as cocoa butter), pessaries, vaginal tabs, foams or enemas.

Compositions suitable for transdermal administration include ointments, gels, patches and injections including powder injections.

Any adaptation(s) and modification(s) should or are intended to be comprehended within the meaning and range of equivalents of the disclosed embodiments.

1. A composition comprising a therapeutic agent chosen from the group:
Apolipoprotein C-II, any active fraction of Apolipoprotein C-II's amino acid sequence.

2. The composition of claim 1, wherein said therapeutic agent is administered via an edible product fortified with the therapeutic agent.

3. The composition of claim 2, wherein said edible product can be chosen from the group: milk, cereal, water, beverages.

4. The composition of claim 1, wherein said therapeutic agent is administered as a drug by a means chosen from the group: oral, parenteral, mucosal, vaginal, rectal, transdermal.

5. The composition of claim 1, wherein said therapeutic agent is used to prevent obesity in mammals.

6. The composition of claim 1, wherein said therapeutic agent is used to treat obesity in mammals.

7. The composition of claim 1, wherein said therapeutic agent is used to bring about non-medical, cosmetic weight loss in mammals.

8. The composition of claim 1, wherein said therapeutic agent is used to prevent a metabolic disorder in mammals.

9. The composition of claim 1, wherein said therapeutic agent is used to treat a metabolic disorder in mammals.

10. The composition of claim 8, wherein said metabolic disorder is chosen from the group: hyperlipidemia, hyperglycemia, diabetes, hypertension, cardiovascular, stroke, gastrointestinal.

11. The composition of claim 9, wherein said metabolic disorder is chosen from the group: hyperlipidemia, hyperglycemia, diabetes, hypertension, cardiovascular, stroke, gastrointestinal.
